(19) United States
(12) Patent Application Publication
(10) Pub. No.: US 2014/0236013 A1
(43) Pub. Date: Aug. 21, 2014

(54) ULTRASOUND DIAGNOSTIC APPARATUS AND ULTRASOUND IMAGE PRODUCING METHOD

(71) Applicant: FUJIFILM Corporation, Tokyo (JP)

(72) Inventor: Yoshiaki SATOH, Ashigara-kami-gun (JP)

(73) Assignee: FUJIFILM Corporation, Tokyo (JP)

(21) Appl. No.: 14/264,519

(22) Filed: Apr. 29, 2014

Related U.S. Application Data
(63) Continuation of application No. PCT/JP2012/078594, filed on Nov. 5, 2012.

(30) Foreign Application Priority Data
Nov. 10, 2011 (JP) 2011-246259
Aug. 20, 2012 (JP) 2012-181453

(51) Int. Cl. A61B 8/08; A61B 8/14; A61B 8/00

(52) U.S. Cl. CPC A61B 8/5207; A61B 8/54; A61B 8/56; A61B 8/145; A61B 8/4483
USPC 600/447

(57) ABSTRACT

An ultrasound diagnostic apparatus controls, out of a plurality of arithmetic cores each for phasing addition on element data, the no river of arithmetic cores to be used in phasing addition on element data in accordance with remaining power of a built in battery, and produces an ultrasound image based on the element data that has been subjected to phasing addition by the arithmetic cores used.

### CROSS REFERENCE TO RELATED APPLICATIONS

This application is a continuation application of International Application PCT/JP2012/078594 filed on Nov. 5, 2012, which claims priority under 35 U.S.C. 119(a) to Application No. 2011-246259 filed in Japan on Nov. 10, 2011 and Application No. 2012-181453 filed in Japan on Aug. 20, 2012, all of which are hereby expressly incorporated by reference into the present application.

### BACKGROUND OF THE INVENTION

The present invention relates to an ultrasound diagnostic apparatus and an ultrasound image producing method and particularly to an ultrasound diagnostic apparatus having a battery to supply power to each component of an ultrasound probe and a diagnostic apparatus body.

Conventionally, ultrasound diagnostic apparatuses using ultrasound images have been employed in the medical field. In general, this type of ultrasound diagnostic apparatus comprises an ultrasound probe having a built-in transducer array and an apparatus body connected to the ultrasound probe. The ultrasound probe transmits an ultrasonic beam toward the inside of a subject's body, receives ultrasonic echoes from the subject, and the apparatus body electrically processes the reception signals to produce an ultrasound image.

In such ultrasound diagnosis, various examinations such as B-mode examination, M-mode examination, CF-mode examination and PW-mode examination are performed. In recent years, an ultrasound diagnostic apparatus capable of those examinations has been reduced in size by adopting an application specific integrated circuit (ASIC) or a processor and is applied as a mobile ultrasound diagnostic apparatus, for example. However, when those various examinations are carried out by a single apparatus, since signal processing, image processing or other processing requires many arithmetic operations, there is a problem that processing speed of the apparatus decreases.

As a technology for improving the processing speed, ultrasound diagnostic apparatuses which perform parallel arithmetic operations using a large number of arithmetic cores in signal processing and the like have been proposed, as disclosed in JP 2006-174902 A.

The apparatus disclosed in JP 2006-174902 A divides a measurement region into plural regions in a scanning line direction, and assigns an arithmetic core to each of the divided regions, whereby image processing is carried out in a parallel fashion in the scanning line direction, and thus the processing speed can be improved.

However, since the apparatus of JP 2006-174902 A is mounted with many arithmetic cores, power consumption thereof increases, and a prolonged use of the apparatus becomes difficult if the apparatus is a mobile ultrasound diagnostic apparatus and runs on a built-in battery.

### SUMMARY OF THE INVENTION

The present invention has been made to solve the problem in the prior art and has an object to provide an ultrasound diagnostic apparatus and an ultrasound image producing method that can improve the processing speed and enables the prolonged operation when a battery is in use.

The ultrasound diagnostic apparatus according to the present invention comprises: a transducer array; a transmission unit configured to transmit an energy beam toward a subject; a reception circuit configured to process a reception signal outputted from the transducer array that received ultrasonic waves generated from the subject upon transmission of the energy beam to thereby generate element data; a plurality of arithmetic cores configured to respectively perform phasing addition on the element data; a built-in battery configured to supply power to the plurality of arithmetic cores; a controller configured to control a number of arithmetic cores out of the plurality of arithmetic cores to be used in phasing addition on the element data in accordance with remaining power of the battery; an image producer configured to produce an ultrasound image based on the element data that has been subjected to phasing addition by the arithmetic cores.

The controller can define a plurality of divisional regions by dividing a measurement region in a depth direction, and assign each of the divisional regions to each of the arithmetic cores. The controller can also define a plurality of divisional regions by dividing a measurement region in a depth direction and a scanning direction, and assign each of the arithmetic cores to each of the divisional regions.

The controller preferably regulates a number of divisions in the depth direction of the measurement region in accordance with the remaining power of the battery so as so reduce the number of arithmetic cores to be used in phasing addition as the remaining power or the battery decreases.

In addition, the controller preferably reduces a depth of the measurement region in accordance with the remaining power of the battery seed that the number of arithmetic cores to be used in phasing addition is reduced as the remaining power of the battery decreases.

The controller preferably switches depth positions of focus points to be used in phasing addition by the arithmetic cores in the respective divisional regions frame by frame, when the remaining power of the battery is lower than a predetermined power.

The controller preferably performs frame-correlation processing on frames in which depth positions of focus points have been switched.

The controller can perform phasing addition on the element data using all of the arithmetic cores when an AC adapter is in use, and reduce the number of arithmetic cores to be used in phasing addition in accordance with reduction in the remaining power of the battery when the battery is in use.

The controller preferably controls the number of arithmetic cores to be used in phasing addition so as to satisfy A=B×S/100 when the battery is in use, having the remaining power of the battery being S (%), the number of arithmetic cores used being A, and the number of arithmetic cores in total being B.

The energy bears is one of an ultrasonic beam and an irradiation light beam.

It is preferable that the ultrasound diagnostic apparatus further comprises a super function unit configured to perform fast Fourier transform under control of the controller.

The method for producing an ultrasound image comprising the steps of: transmitting an energy beam toward a subject; receiving ultrasonic waves generated from the subject by transmitting the energy beam in a transducer array; generating element data by processing in a reception circuit reception signals outputted from the transducer array that received the ultrasonic wares; controlling a number of arithmetic cores to be used in phasing addition on the element data out of a plurality of arithmetic cores respectively for phasing addition on the element data in accordance with remaining power of a battery for supplying power to the plurality of arithmetic cores.

According to the present invention, since the number of arithmetic cores used in phasing addition of element data is regulated in accordance with the remaining power of the battery, the processing speed can be improved, and the prolonged operation of the apparatus when the battery is in use becomes possible.

### BRIEF DESCRIPTION OF THE DRAWINGS

FIG. 1 is a block, diagram illustrating a configuration of an ultrasound diagnostic apparatus according to Embodiment 1 of the invention.

FIG. 2 is a schematic view illustrating a measurement region in an ultrasound diagnosis.

FIG. 3 is a schematic view of element data stored in an element memory.

FIG. 4 is a flow chart illustrating the operation in Embodiment 1.

FIG. 5 is a flow chart illustrating an examination mode in Embodiment 1.

FIG. 6 is a schematic view illustrating focus points on a measurement line when the number of arithmetic cores to be used is set to 40.

FIG. 7 is a schematic view illustrating now focus points on a measurement line are replaced in each frame when the number of the arithmetic cores to be used is set to 20.

FIG. 8 is a schematic view illustrating how focus points on a measurement line ere replaced in each frame when the number of the arithmetic cores to be used is set to 10.

FIG. 9 is a block diagram illustrating a configuration of an ultrasound diagnostic apparatus according to Embodiment 2.

### DETAILED DESCRIPTION OF THE INVENTION

Embodiments of the present invention will be described below based on the appended drawings.

### Embodiment 1

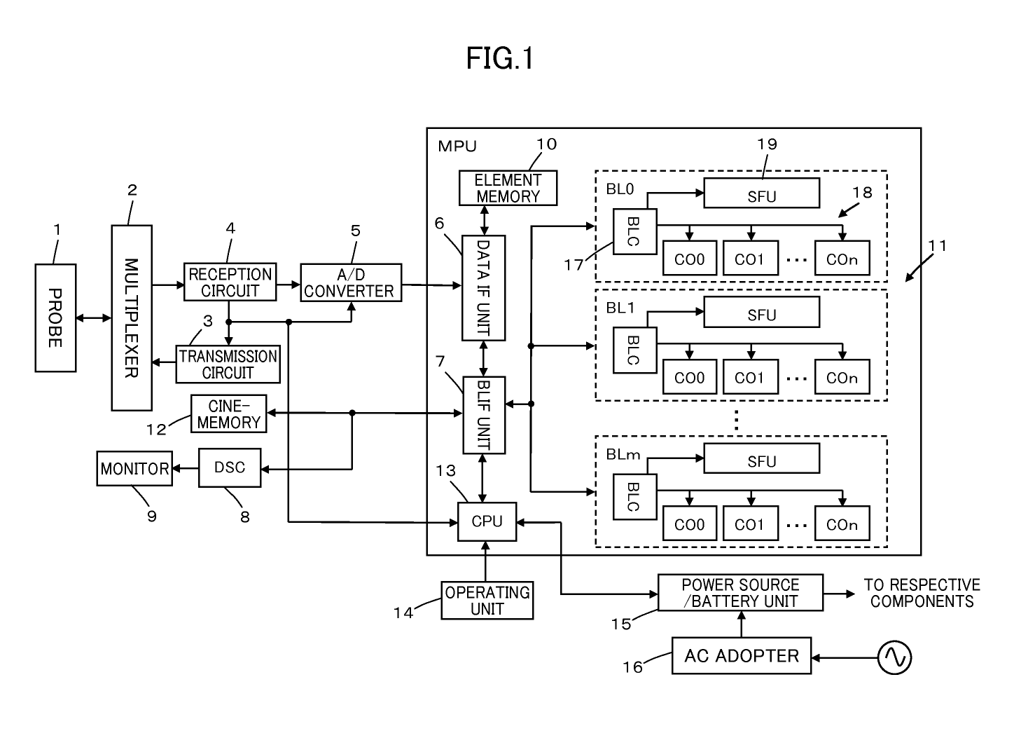

FIG. 1 illustrates a configuration of an ultrasound diagnostic apparatus according to Embodiment 1 of the invention. The ultrasound diagnostic apparatus includes a probe 1, which is connected to a transmission circuit 3 and a reception circuit 4 via a multiplexer 2. The reception circuit 4 is connected to an A/D converter 5, a data interface (IF) unit 6, a block interface (BLIF) unit 7, a digital scan converter (DSC) 8 and a monitor 9 in order, and the data IF unit 6 is connected to an element memory 10, while the BLIF unit 7 is connected to a signal processor 11 and a cine-memory 12.

The transmission circuit 3, the reception circuit 4, the A/D converter 5 and the BLIF unit 7 are connected to a CPU 13. The CPU 13 is connected to an operating unit 14 and to a power source/battery unit 15, and the power source/battery unit 15 is connected to an AC adaptor 16.

The probe 1 includes a transducer array in which a plurality of transducer elements are arranged one-dimensionally or two-dimensionally. These transducer elements each transmit ultrasonic waves according to driving signals supplied from the transmission circuit 3 via the multiplexer 2 and receive ultrasonic echoes from the subject to output reception signals. Each of the transducer elements comprises a vibrator composed of a piezoelectric body and electrodes each provided on both ends of the piezoelectric body. The piezoelectric body is composed of, for example, a piezoelectric ceramic represented by a lead zirconate titanate (PZT), a piezoelectric polymer, represented by polyvinylidene fluoride (PVDF), or a piezoelectric monochristal represented by lead magnesium niobate lead titanate solid solution (PMN-PT).

When the electrodes of each of the vibrators ate supplied with a pulsed voltage or a continuous-wave voltage, the piezoelectric body expands and contracts to cause the vibrator to produce pulsed or continuous ultrasonic waves. These ultrasonic waves are combined to form an ultrasonic beam (energy beam). Open reception of propagating ultrasonic waves, each vibrator expands and contracts to produce an electric signal, which is then outputted as reception signal of the ultrasonic waves.

The multiplexer 2 selects transducer elements to be used in a single transmission, connects the selected transducer elements to the transmission circuit 3 at the timing of transmission, and connects the transducer elements to the reception circuit 4 at the timing of reception.

The transmission circuit 3 includes, for example, a plurality of pulsers and adjusts the delay amounts for driving signals based on a transmission delay pattern selected according to an instruction signal transmitted from the CPU 13 so that the ultrasonic waves transmitted from a plurality of arrayed transducers of the probe 1 form an ultrasonic beam and supplies the arrayed transducers with delay-adjusted driving signals.

In accordance with the instruction signal from the CPU 13, the reception circuit 4 amplifies reception signals transmitted from the respective elements of the transducer array.

In accordance with the instruction signal from the CPU 13, the A/D converter 5 performs A/D conversion on the reception signal amplified in the reception circuit 4 to generate element data. Under the control of the CPU 13, the data IF unit 6 communicates between the A/D converter 5 and the element memory 10 or between the element memory 10 and the BLIF unit 7. The element memory 10 stores element data generated in the A/D converter 5 sequentially via the data IF unit 6. Under the control of the CPU 13, the BLIF unit 7 communicates between the signal processor 11 and the data IF unit 6, the cine-memory 12 or the DSC 8.

The signal processor 11 comprises a plurality of blocks (BL0 to BLm) connected in parallel to the BLIF unit 7, each of the blocks includes a block controller (BLC) 17 connected to the BLIF unit 7, and the BLC 17 is connected to a plurality of arithmetic cores (CO0 to COn) 18 and a super function unit (SFU) 19. The signal processor 11 produces a scanning line signal (sound ray signal) in which focuses of the ultrasonic echo are concentrated, and in particular a plurality of scanning line signals in a measurement region are produced by being shared with the assigned blocks. The plurality of arithmetic cores 18 respectively perform phasing addition on each element data under the control of the BLC 17. The SFU 19 performs arithmetic operations such as fast Fourier transform (FFT) and trigonometric operations. The BLC 17 controls the arithmetic operations by the plurality of arithmetic cores 18 and the SFU 19 to thereby control production of scanning line signals in each of the blocks.

The DSC 8 converts the scanning line signals produced in the signal processor 11 into image signals compatible with an ordinary television signal scanning mode (raster conversion).

The monitor 9 includes a display device such as an LCD, for example, and displays an ultrasound diagnostic image based on the image signals produced by the DSC 8.

The CPU 13 controls the components in the ultrasound diagnostic apparatus according to the instruction entered by an operator using the operating unit 14. The CPU 13 checks the remaining power of the power source/battery unit 15 and controls the signal processor 11 according to the remaining power.

The operating unit 14 is provided for the operator to perform input operations and may be composed of, for example, a keyboard, a mouse, a track ball, and/or a touch panel.

The AC adapter 16 supplies the power source/battery unit 15 with power from a commercial power source. The power source/battery unit 15 supplies the components in the ultrasound diagnostic apparatus with power. When the AC adapter 16 is not connected, the built-in battery supplies the components with power.

The controller in the present invention comprises the CPU 13 and the BLCs 17 in the respective blocks, and the image producer in the present invention comprises the DSC 8 and the monitor 9.

Next, described is how the element data obtained through transmission and reception of the ultrasonic beam is processed.

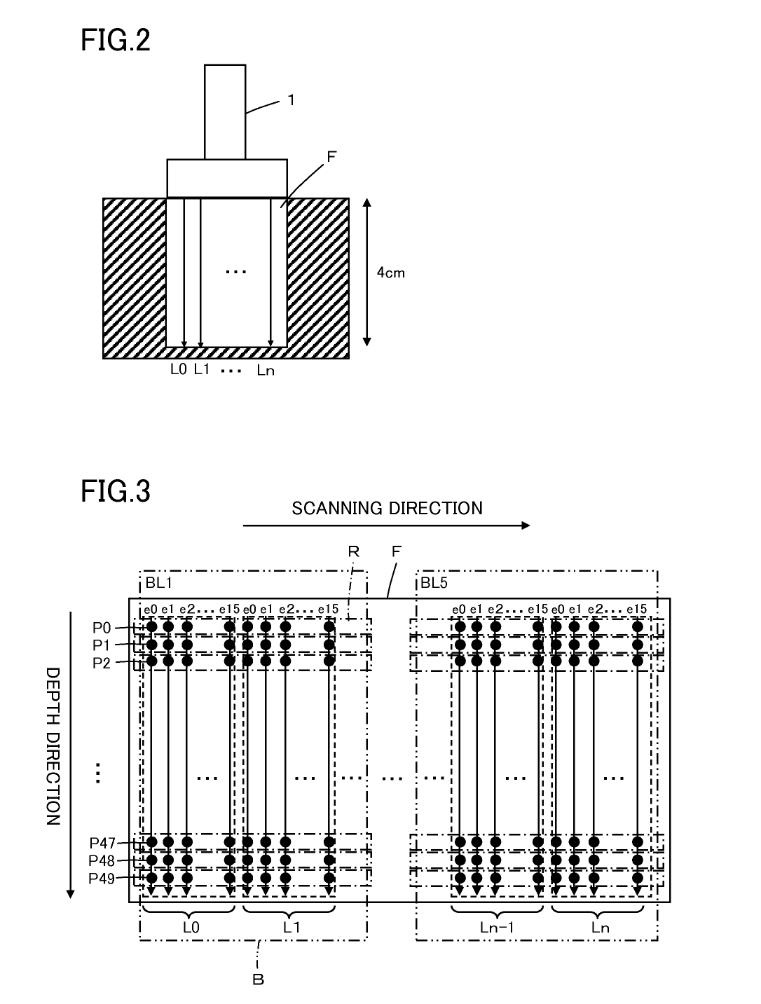

As illustrated in FIG. 2, the transducer array in the probe 1 sequentially transmits ultrasonic beams toward the subject, fox example, using 16 elements for a single transmission, receives ultrasonic echoes from a predetermined measurement region F and outputs reception signals, each of which is subjected to A/D conversion to obtain the element data. At this time, focus points P0 to P49 are placed in the measurement region F at the depths respectively at which the measurement region F is divided into 50 regions in the depth direction, and the ultrasonic beams are sequentially transmitted from and received by the respective elements so as to form the scanning lines L0 to Ln each of which includes the focus points P0 to P49. As illustrated in FIG. 3, the obtained element data e0 to e15 corresponding to the focus points P0 to P41 in the measurement region F is sequentially stored in the element memory 10 for each of the scanning lines L0 to Ln.

The element data stored in the element memory 10 is divided in the scanning direction, separately inputted in each block of the signal processor 11 and processed therein. In an example where the signal processor 11 comprises 5 blocks (BL1 to BL5), and each of the blocks comprises 50 arithmetic cores CO0 to CO49 constituting the arithmetic core 18, the BLC 17 in each of the blocks divides the measurement region F into 5 regions (regions B) in the scanning direction and into 50 regions in the depth direction so as to form 250 divisional regions R, the element data included in each of the 5 regions B (element data of n/5 the scanning lines) is inputted in each of the blocks BL1 to BL5, and in each region B, each of the divisional regions R is assigned to one arithmetic core 18 of one block. At this time, the respective depths at which the measurement region F is divided into 50 regions in the depth direction correspond to the depth positions where the focus points P0 to P49 of the ultrasonic beam are placed, and the divisional regions R respectively include the focus points P0 to P49.

The arithmetic cores 18 that are respectively assigned to the divisional regions R in this manner perform signal processing on the element data in each of the blocks in the depth direction in parallel and perform signal processing on the element data in the plural blocks in the scanning direction in parallel. Specifically, 50 arithmetic cores 18 in each of the blocks perform phasing addition on the element data corresponding to the respective focus points in the assigned divisional regions R in the depth direction in parallel. Similar processing is performed in the plural blocks in the scanning direction in parallel. The element data subjected to phasing addition by the arithmetic cores 18 is then subjected to matching addition by the BLC 17 in each of the blocks.

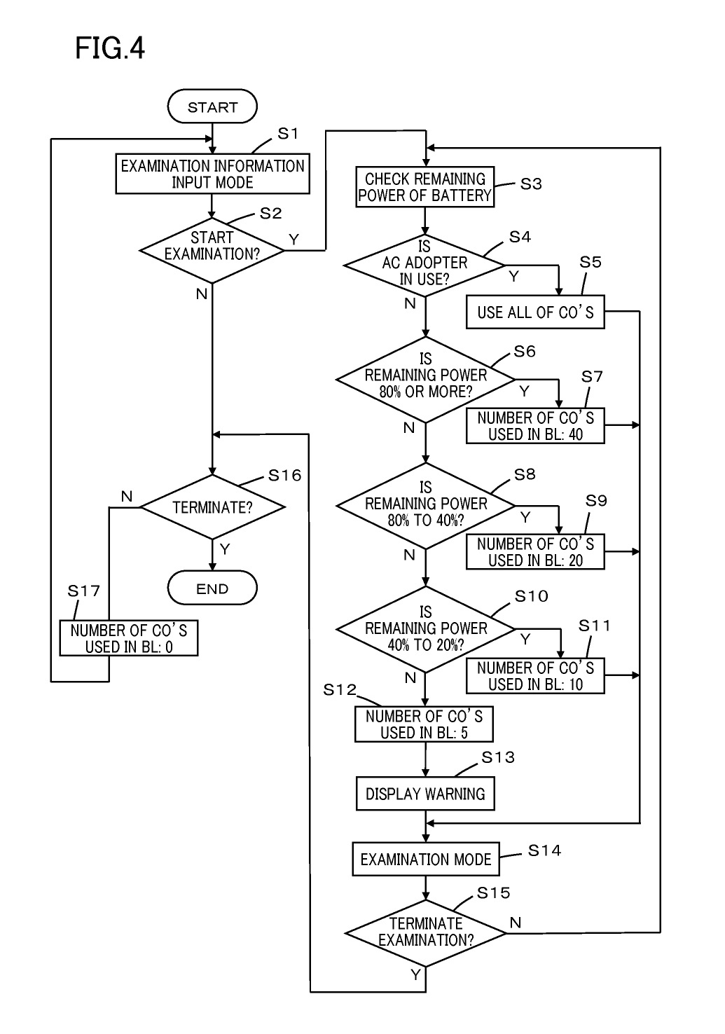

Next, the operation of Embodiment 1 will be described referring to the flowchart of FIG. 4.

First, once examination information including the patient information and an examination request is entered from the operating unit 14 in the examination information input mode in Step S1, the CPU 13 waits for an instruction from an operator to start the examination in Step S2. When the instruction to start the examination is entered through the operating unit 14, then the operation proceeds to Step S3, where the CPU 13 checks the remaining power of the power source/battery unit 15 and confirms whether the AC adapter 16 is in use in Step S4. On the other hand, when the instruction to start the examination is not entered, the operation proceeds to Step S16 and waits for an instruction to terminate the examination.

If the AC adapter 16 is in use in Step S4, the operation proceeds to Step S5 where the setting is made in the signal processor 11 such that the BLC 17 in each of the blocks performs signal processing using ail the arithmetic cores 18. In particular, as illustrated in FIG. 3, the measurement region F is divided into 5 regions in the scanning direction and also into 50 regions, in the depth region to form 250 divisional regions R, the 5 regions B are respectively assigned to the blocks BL1 to BL5, and each of the divisional regions R is assigned to one arithmetic core 18 in each of the blocks.

When the AC adapter is not in use in Step S4, the operation proceeds to Step S6, and if the remaining power of the battery is 80% or more, the operation proceeds to Step S7 where the number of arithmetic cores to be used for phasing addition in each of the blocks is set to 40. At this time, the divisional regions R are defined by dividing the measurement region F into 5 regions in the scanning direction and also into 40 regions in the depth direction, while the 5 regions B are respectively assigned to the blocks BL1 to BL5, and each of the divisional regions R is assigned to one arithmetic core 18 in each block.

If the remaining power of the battery is less than 80% in Step S6, the operation proceeds to Step S8 where, if the remaining power of the battery is less than 80% but not less than 40%, the number of arithmetic cores to be used for phasing addition in each of the blocks is set to 20. At this time, the divisional regions R are defined by dividing the measurement region F into 5 regions in the scanning direction and also into 20 regions in the depth region, while the 5 regions B are respectively assigned to the blocks BL1 to BL5, and each of the divisional regions R is assigned to one arithmetic core 18 in each block.

If the remaining power of the battery is less than 40% in Step S8, the operation proceeds to Step S10 where, if the remaining power of the battery is less than 40% but not less than 20%, the number of arithmetic cores to be used for phasing addition in each of the blocks is set to 10 in Step S11. At this time, the divisional regions R are defined by dividing the measurement region F into S regions in the scanning direction and also into 10 regions in the depth region, while the 5 regions B are respectively assigned to the blocks BL1 to BL5, and each of the divisional regions R is assigned to one arithmetic core 18 in each block.

Furthermore, if the remaining power of the battery is less than 20% in Step S10, the operation proceeds to Step S12 where, similarly to the above steps, the number of arithmetic cores to be used for phasing addition in each of the blocks is set to 5, and a warning for the lower remaining power of the battery is displayed for the operator in Step S13.

As described above, once the number of arithmetic cores to be used for phasing addition in each of the blocks is set in Steps S5, S7, S9, S11 and S12, the operation proceeds to Step S14 to execute the examination mode and thereafter waits for an instruction from the operator to terminate the examination in Step S15. If an instruction not to terminate but to continue the examination is entered in Step S15, the operation returns to Step S3 and checks again the remaining power of the battery.

On the other hand, if an instruction to terminate the examination is entered, the operation proceeds to Step S16 and waits for an instruction to terminate a series of examination processing. When an instruction to terminate the examination is entered, the examination processing is terminated as is, whereas when an instruction to continue the examination is entered, the number of arithmetic cores to be used for phasing addition in each of the blocks is set to 0, and the operation returns to Step S1 to accept a new input of examination information.

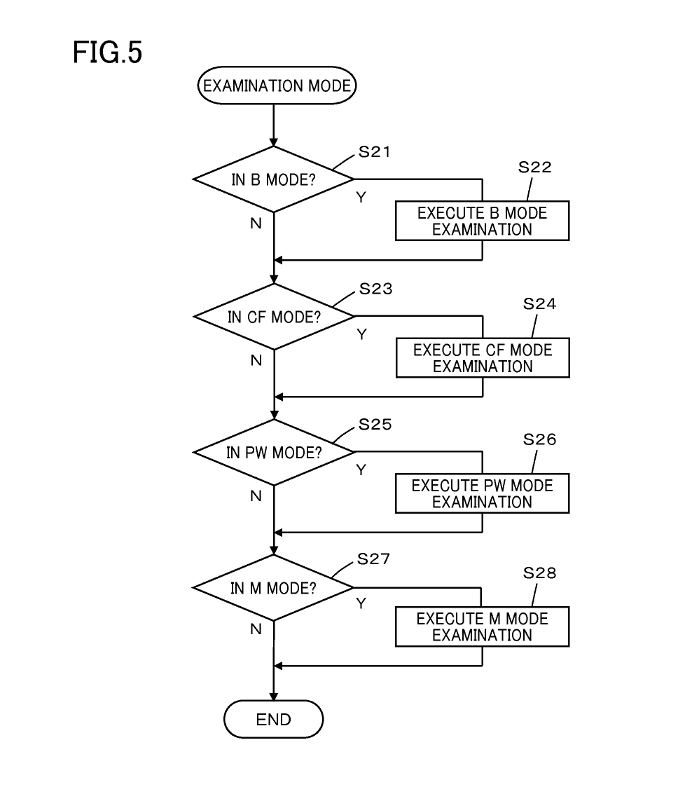

In the examination mode in Step S14, one or more of previously set examination modes such as brightness mode (B mode), color flow mode (CF mode), pulsed wave mode (PW mode), and motion mode (M mode) as shown in FIG. 5 may be selected to execute ultrasound diagnosis. That is, the CPU 13 checks examination information entered in Step S1 to determine which mode has been designated and, upon verifying designation of B mode in Step S21, the operation proceeds to Step S22 to execute examination in B mode. Upon verifying designation of CF mode in Step S23, the operation proceeds to Step S24 to execute examination in PW mode. Upon verifying designation of PW mode in Step S25, the operation proceeds to Step S26 to execute examination in PW mode. Upon verifying designation of M mode in Step S27, the operation proceeds to Step S38 to execute examination in M mode.

The examinations in Steps S22, S24, S26 and S18 described above are executed based on the setting for the number of arithmetic cores to be used made in Steps S5, 7, 9, 11 and 12.

In particular, when the setting is made in Step S5 to use all the arithmetic cores 18 in each of the blocks for signal processing, focus points P0 to P49 of the ultrasonic beam are placed at the depths respectively at which the measurement region F is divided into 50 regions in the depth direction, similarly to the divisional regions R formed in Step S5, and the elements transmit and receive the ultrasonic beams as illustrated in FIG. 2. Accordingly, the element data as shown in FIG. 3 is obtained, the obtained element data of each region B of the 5 regions divided in the scanning direction is inputted in each of the blocks, and one divisional region R is assigned to one arithmetic core 18 in the each of the blocks, whereby 50 divisional regions R for each of the blocks are subjected to phasing addition in parallel. In this manner, 250 divisional regions R formed by dividing the measurement region F into 5 regions in the scanning direction end also into 50 regions in the depth direction are subjected to phasing addition in parallel, so that the speed of signal processing can be improved.

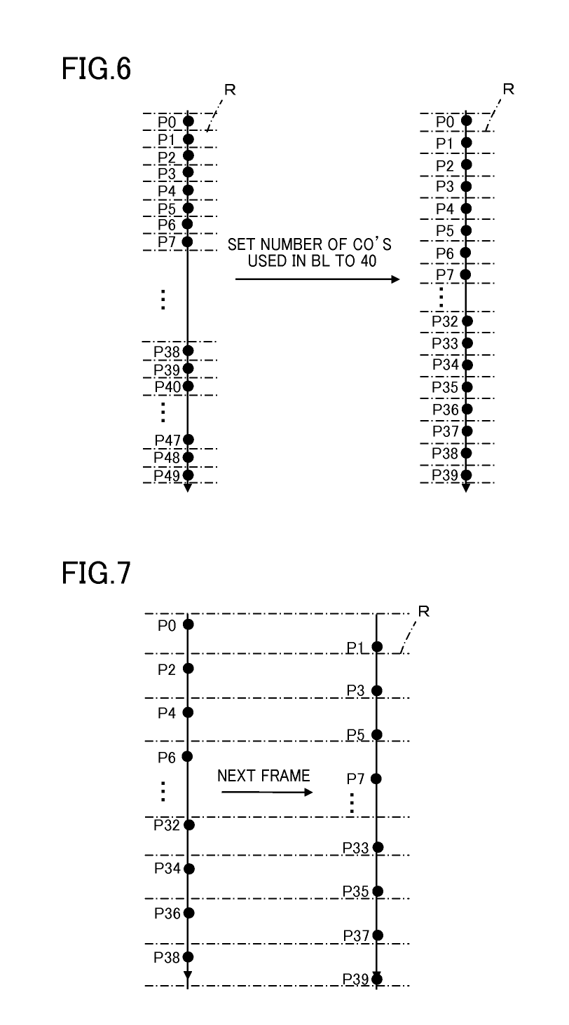

In addition, when the number of arithmetic cores to be used for phasing addition in each of the blocks is set to 40 in Step S7, focus points P0 to P39 of the ultrasonic beam are placed at the depths respectively at which the measurement region F is divided into 40 regions in the depth direction, similarly to the divisional regions R formed in Step S7, and the elements transmit and receive the ultrasonic beams. Accordingly, the ultrasonic beams are transmitted and received such that the focus points P0 to P39 are included respectively in the divisional regions R. The obtained element data of each region B of the 5 regions divided in the scanning direction is inputted in each of the blocks, and one divisional region R is assigned to one arithmetic core 18 in the each of the blocks, whereby the element data is subjected to phasing addition in the respective blocks in parallel. Accordingly, as Illustrated in FIG. 6, compared to the setting made in Step S5 in which the measurement region F is subjected to signal processing by using 50 arithmetic cores 18, since the same measurement region F is subjected to signal processing by using 40 arithmetic cores, 10 arithmetic cores can be saved from operating. Therefore, power consumption in signal processing can be reduced to four fifths of that of the setting in Step S5.

In addition, when the number of arithmetic cores to be used for phasing addition in each of the bloc he is set to 20 in Step S9, focus points P0 to P39 arc given to the ultrasonic beam at the depths respectively at which the measurement region F is divided into 40 regions in the depth direction, similarly to the setting in Step S7, and the elements transmit and receive the ultrasonic beams. On the other hand, the divisional regions R are defined by dividing the measurement region F into 20 reasons in the depth direction. Hence, the ultrasonic beams are transmitted and received such that two of the focus points P0 to P39 are included in each of the divisional regions R. The obtained element data of each region B of the 5 regions divided in the scanning direction is inputted in each of the blocks, and one divisional region R is assigned to one arithmetic core 18 in the each of the blocks, whereby the element data is subjected to phasing addition in the respective blocks in parallel. At this time, as illustrated in FIG. 7, the arithmetic cores perform phasing addition on the element data for every two frames, while replacing one of the two focus points included in each of the divisional regions R with another frame by frame. More specifically, the arithmetic cores perform phasing addition on the element data based on one series of the focus points P0, P2, . . . and P38 respectively included in the divisional regions R and thereafter perform phasing addition on the element data in the next frame based on the other series of the focus points P1, P3, . . . and P39 also included in the respective divisional regions R. Accordingly, compared to the setting made in Step S5 in which the measurement region F is subjected to signal processing by using 50 arithmetic cores 18, since the same measurement region F is subjected to signal processing by using 20 arithmetic cores, 30 arithmetic cores can be saved from operating. Therefore, power consumption in signal processing can be reduced to two fifths of that of the setting in Step S3.

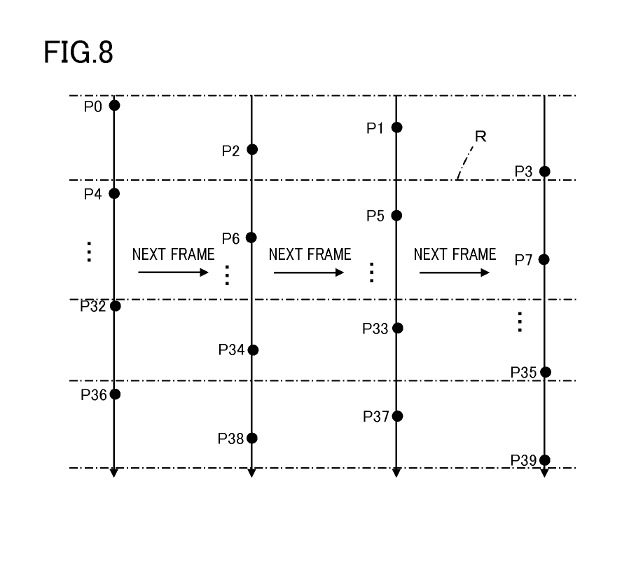

In addition, when the number of arithmetic cores to be used for phasing addition in each of the blocks is set to 10 in Step S11, focus points P0 to P39 of the ultrasonic beam are placed at the depths respectively at which the measurement region F is divided into 40 regions in the depth direction, similarly to the setting in Step S7, and the elements transmit and receive the ultrasonic beams. On the other hand, the divisional regions R are defined by dividing the measurement region F into 10 regions in the depth direction. Hence, the ultrasonic beams are transmitted and received such that four of the focus points P0 to P39 are included in each of the divisional regions R. The obtained element data of each region B of the 5 regions divided in the scanning direction is inputted in each of the blocks, and one divisional region R is assigned to one arithmetic core 18 in the each of the blocks, whereby the element data is subjected to phasing addition in the respective blocks in parallel. At this time, as illustrated in FIG. 8, the arithmetic cores perform phasing addition on the element data for every four frames, while replacing each of the four focus points included in the divisional region R with another sequentially frame by frame. More specifically, the arithmetic cores perform, phasing addition on the element data based on one series of the focus points P0, P4 . . . and P36 respectively included in the divisional regions R, then perform phasing addition on the element data in the second frame based on the second series of focus points P2, P6, . . . and P38, perform phasing addition on the element data in the third frame based on the third series of focus points P1, P5, . . . and P37, and further perform phasing addition on the element data in the fourth, frame based on the fourth series of focus points P3, P7, . . . and P39. Accordingly, compared to the setting made in Step S5 in which the measurement region F is subjected to signal processing by using 50 arithmetic cores 18, since the same measurement region F is subjected to signal processing by using 10 arithmetic cores, 40 arithmetic cores can be saved from operating. Therefore, power consumption in signal processing can be reduced to one fifths of that of the setting in Step S5.

In this manner, once the number of arithmetic cores 18 to be used is set in Steps S9 and S11, the BLC 17 of the signal processor 11 switches the depth positions of focus points for phasing addition using the arithmetic cores in each of the divisional regions frame by frame, whereby the number of focus points in the depth direction can be maintained at 40 in every two frames when the number of used arithmetic cores is 40 or in every four frames when the number of used arithmetic cores is 20. By performing correlation processing on frames, generation of flicker in which focus points are shifted frame by frame can be suppressed.

As described above, the signal processing of the element data is executed by the signal processor 11 while controlling the number of used arithmetic cores in accordance with the remaining power of the battery, and the scanning line signals in which focuses of the ultrasonic echo are concentrated are produced. Subsequently, the scanning line signals produced by the signal processor 11 are outputted to the DSC 8 through the BLIF unit 7 to be converted to image signals, which are then out punted to the monitor 9 so that the ultrasound diagnostic image is displayed.

As described above, examinations in the respective modes are executed in accordance with the remaining power of the battery, and the operation proceeds to Step S15 shown in FIG. 4 to verify the completion of examination based on the examination information for the present round of examination.

According to this embodiment, the measurement region F is divided in the scanning direction and the depth direction into a plurality of divisional regions R, and the respective divisional regions R are subjected to signal processing in parallel. Thus, the speed of signal processing can be improved. In addition, since the number of arithmetic cores used is reduced as the remaining power of the battery decreases by controlling the number of divisions of the measurement region F in the depth direction in accordance with the remaining power of the battery, the operating time of the ultrasound diagnostic apparatus can be prolonged while the processing speed is maintained.

In the above-described embodiment, the signal processor 11 comprising a plurality of blocks performs signal processing on the element data by dividing the measurement region F in the scanning direction. However, the present invention is not limited thereto, as long as a plurality of divisional regions R that are divisions of the measurement region F in the depth direction can be formed. In other words, the signal processor 11 may be composed of a single block, one arithmetic core 18 is assigned to each or the divisional regions R that are divisions of the measurement region F in the depth direction, and phasing addition may be performed on the element data in the respective divisional regions R in parallel.

Moreover, in the above-described embodiment, the number of arithmetic cores used is altered from 50 to 40, 20, and 10 in a stepwise fashion in accordance with the remaining power of the battery, but the present invention is not limited thereto. For example, the BLC 17 of the signal processor 11 may control the number of used arithmetic cores so as to satisfy A=B×S/100 when the battery is in use, having the remaining power of the battery being S(%), the number of used arithmetic cores being A, and the number of total arithmetic cores being B.

Further, in the above-described embodiment, the element data of each region B of the measurement region F is inputted in each of the blocks of the signal processor 11, but the present invention is not limited thereto, as long as the signal processor 11 can perform signal processing in accordance with the remaining power of the battery. For example, while the element data for the respective regions B is inputted into five blocks in the signal processor 11, and phasing addition is performed by using 40 arithmetic cores in each of the blocks in the setting in Step S7, the element data may be inputted into four blocks, and phasing addition may be performed by using 50 arithmetic cores in each of the blocks, saving the remaining one block from operating. Similarly, in Step S9, the element data may be inputted into two blocks, and phasing addition may be performed by using 50 arithmetic cores in each of the blocks, saving the remaining three blocks from operating. In Step S11, the element data may be inputted into a single block, and phasing addition may be performed by using 50 arithmetic cores in the block, saving the remaining four blocks from operating.

In addition, In the above-described embodiment, compared to the setting in Step S5 in which the measurement region F is divided into 50 regions in the depth direction, the measurement region F is divided into 40 regions in the depth direction in Step S7. However, the present invention is not limited thereto, as long as the measurement region F can be divided into 40 regions in the depth direction. For example, compared to the setting in Step S5 in which the measurement region F is divided into 50 regions in the depth direction, the setting in Step S7 may allow the measurement region F to he reduced by 10 divisional regions. In this manner, the focus points can be maintained to have the same intervals as those in the setting in Step S7, preventing reduction in the image quality.

### Embodiment 2

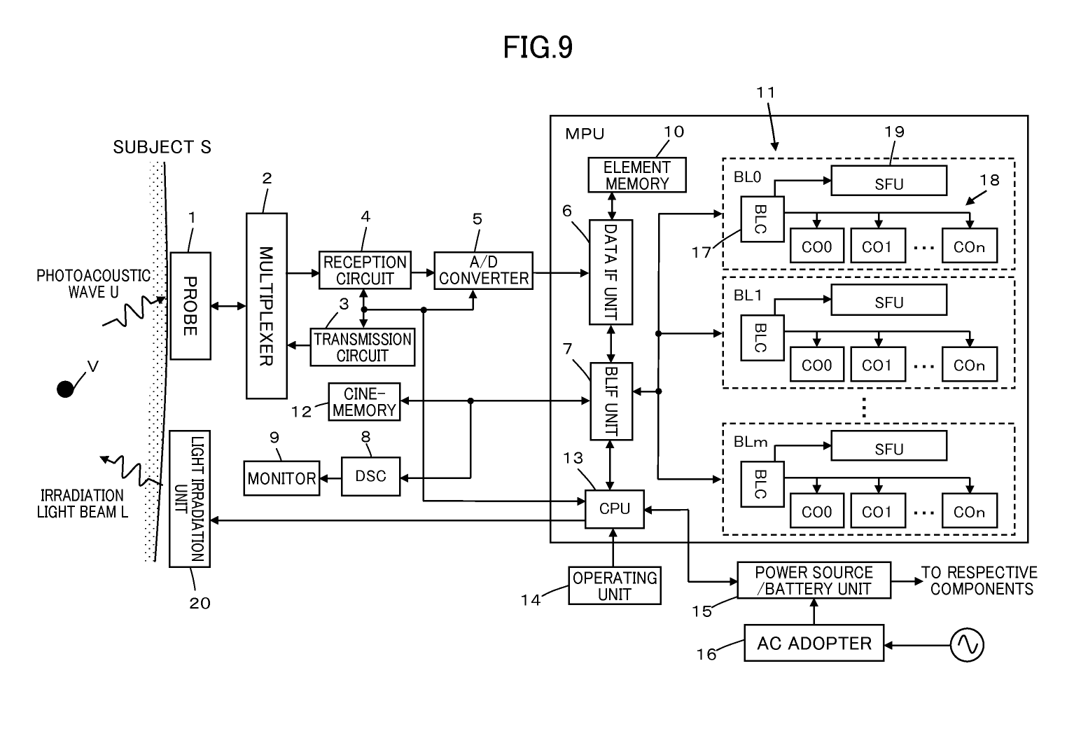

FIG. 9 illustrates a configuration of an ultrasound diagnostic apparatus according to Embodiment 2. The ultrasound diagnostic apparatus performs so-called photoacoustic imaging (PAI) to image the Inside of the subject S using the photoacoustic effect. In the ultrasound diagnostic apparatus in Embodiment 1 as illustrated in FIG. 1, a light irradiation unit 20 is additionally connected to the CPU 13.

The light irradiation unit 20 sequentially emits plural irradiation light beams (energy beams) L having different wavelengths from one another toward the subject S and comprises a semiconductor laser (LD), a light emitting diode (LED), a solid laser, a gas laser or the like. The light irradiation unit 20 uses, for example, pulsed laser light beams as irradiation light beams L and emits pulsed laser light beams toward the subject S, while sequentially changing wavelengths for pulses.

For photoacoustic imaging, the CPU 13 controls the light irradiation unit 20 to cause the light irradiation unit 20 to emit irradiation light beams L toward the subject S. Once a predetermined living tissue V inside the subject S is irradiated with the irradiation light beams L emitted from the light irradiation unit 20, the lining tissue V absorbs light energy of the irradiation light beams L to thereby release photoacoustic waves U (ultrasonic waves) that are elastic waves.

For example, the irradiation light beam L having a wavelength of about 750 nm and the irradiation light beam L having a wavelength of about 800 nm are emitted from the light irradiation unit 20 sequentially toward the subject S. In the meantime, oxygenated hemoglobin (hemoglobin combined with oxygen; oxy-Hb) included in plenty in a human artery has a higher coefficient of molecular absorption for the irradiation light beam L with a wavelength of 750 nm than for the irradiation light beam L with a wavelength of 800 nm. On the other hand, deoxygenated hemoglobin (hemoglobin not combined with oxygen; deoxy-Hb) included in plenty in a human vein has a lower coefficient of molecular absorption for the irradiation light beam L with a wavelength of 750 nm than for the irradiation light beam L with a wavelength of 800 nm. Accordingly, if an artery and a vein are irradiated with the irradiation light beams L respectively having wavelengths of 800 nm and 750 nm, photoacoustic waves u having intensities corresponding to the respective coefficients of molecular absorption of the artery and the vein will be released.

The photoacoustic waves U released from an artery or a vein, for example, as described above are received by the transducer array arranged in the probe 1 bike in Embodiment 1, and the signal processor 11 performs signal processing using the arithmetic cores 18 based on the reception signal thereof. The number of the arithmetic cores to be used for signal processing in the signal processor 11 is set depending on whether the AC adapter 16 is in use or in accordance with the remaining power of the power source/battery unit 15. The signal processor 11 can perform signal processing in accordance with difference in the intensity of reception signals from liming tissues V and produce a photoacoustic image (ultrasound image) in which each of the living tissues V is imaged.

The photoacoustic image is preferably displayed together with an ultrasound image obtained through transmission and reception of ultrasonic waves by the probe 1. The CPU 13 controls the transmission circuit 3 and the light irradiation unit 20, respectively, to transmit ultrasonic waves from the probe 1 and emit irradiation light beams L from the light irradiation unit 20 sequentially, whereby an ultrasound image and a photoacoustic image can be simultaneously displayed. In a preferable example, the CPU 13 controls the transmission circuit 3 and the light irradiation unit 20 such that a photoacoustic image of one frame is produced during generation of ultrasound images of 10 frames.

According to this embodiment, since a photoacoustic image can be produced In addition to an ultrasound image, a multifaceted observation of a subject can be realized, enabling a detailed diagnosis.

What is claimed is:

1. An ultrasound diagnostic apparatus comprising:
a transducer array;
a transmission unit configured to transmit an energy beam toward a subject;
a reception circuit configured to process a reception signal outputted from the transducer array that received ultrasonic waves generated from the subject upon transmission of the energy beam to thereby generate element data;
a plurality of arithmetic cores configured each to perform phasing addition on the element data;
a battery configured to supply power to the plurality of arithmetic cores;
a controller configured to control, out of the plurality of arithmetic cores, a number of arithmetic cores to be used in phasing addition on the element data in accordance with remaining power of the battery; and
an image producer configured to produce an ultrasound image based on the element data that has been subjected to phasing addition by the arithmetic cores.

2. The ultrasound diagnostic apparatus according to claim 1, wherein the controller defines a plurality of divisional regions by dividing a measurement region in a depth direction, and assigns each of the divisional regions to each of the arithmetic cores.

3. The ultrasound diagnostic apparatus according to claim 1, wherein the controller defines a plurality of divisional regions by dividing a measurement region in a depth direction and a scanning direction, and assigns each of the divisional regions to each of the arithmetic cores.

4. The ultrasound diagnostic apparatus according to claim 2, wherein the controller regulates a number or divisions in the depth direction of the measurement region in accordance with the remaining power of the battery so as to reduce the number or arithmetic cores to be used in phasing addition as the remaining power of the battery decreases.

5. The ultrasound diagnostic apparatus according to claim 3, wherein the controller regulates a number of divisions in the depth direction of the measurement region in accordance with the remaining power of the battery so as to reduce the number of arithmetic cores to be used in phasing addition as the remaining power of the battery decreases.

6. The ultrasound diagnostic apparatus according to claim 2, wherein the controller reduces a depth of the measurement region in accordance with the remaining power of the battery such that the number of arithmetic cores to be used in phasing addition is reduced as the remaining power of the battery decreases.

7. The ultrasound diagnostic apparatus according to claim 3, wherein the controller reduces a depth of the measurement region in accordance with the remaining power of the battery such that the number of arithmetic cores to be used in phasing addition is reduced as the remaining power of the battery decreases.

8. The ultrasound diagnostic apparatus according to claim 4, wherein the controller switches depth positions of focus points to be used in phasing addition by the arithmetic cores in the respective divisional regions frame by frame, when the remaining power of the battery is lower than a predetermined power.

9. The ultrasound diagnostic apparatus according to claim 5, wherein, the controller switches depth positions of focus points to be used in phasing addition by the arithmetic cores in the respective divisional regions frame by frame, when the remaining power of the battery is lower than a predetermined power.

10. The ultrasound diagnostic apparatus according to claim 8, wherein the controller performs frame-correlation processing on frames in which depth positions of focus points have been switched.

11. The ultrasound diagnostic apparatus according to claim 9, wherein the controller performs frame-correlation processing on frames in which depth positions of focus points have been switched.

12. The ultrasound diagnostic apparatus according to claim 4, wherein the controller performs phasing addition on the element data using all of the arithmetic cores when an AC adapter is in use, and reduces the number of arithmetic cores to be used in phasing addition in accordance with reduction in the remaining power of the battery when the battery is in use.

13. The ultrasound diagnostic apparatus according to claim 5, wherein the controller performs phasing addition on the element data using all of the arithmetic cores when an AC adapter is in use, and reduces the number of arithmetic cores to be used in phasing addition in accordance with reduction in the remaining power of the battery when the battery is in use.

14. The ultrasound diagnostic apparatus according to claim 8, wherein the controller controls the number of arithmetic cores to be used in phasing addition so as to satisfy A=B×S/100 when the battery is in use, where the remaining power of the battery is S (%), the number of arithmetic cores used is A, and a number of arithmetic cores in total is B.

15. The ultrasound diagnostic apparatus according to claim 9, wherein the controller controls the number of arithmetic cores to be used in phasing addition so as to satisfy A=B×S/100 when the battery is in use, where the remaining power of the battery is S (%), the number of arithmetic cores used is A, and a number of arithmetic cores in total is B.

16. The ultrasound diagnostic apparatus according to claim 1, wherein the energy beam is one of an ultrasonic beam and an irradiation light beam.

17. The ultrasound diagnostic apparatus according to claim 2, wherein the energy beam is one of an ultrasonic beam and an irradiation light beam.

18. The ultrasound diagnostic apparatus according to claim 3, wherein the energy beam is one of an ultrasonic beam and an irradiation light beam.

19. The ultrasound diagnostic apparatus according to claim 1, further comprising a super function unit configured to perform fast Fourier transform under control of the controller.

20. A method for producing an ultrasound image comprising the steps of:
transmitting an energy beam toward a subject;
receiving in a transducer array ultrasonic waves generated from the subject upon transmission of the energy beam;
generating element data by processing in a reception circuit reception signals outputted from the transducer array that received the ultrasonic waves;
controlling, out of a plurality of arithmetic cores each for phasing addition on the element data, a number of arithmetic cores to be used in phasing addition on the element data in accordance with remaining power of a battery for supplying power to the plurality of arithmetic cores; and
producing an ultrasound image based on the element data that has undergone phasing addition by the arithmetic cores.
